(19) United States
(12) Patent Application Publication
(10) Pub. No.: US 2008/0226758 A1
(43) Pub. Date: Sep. 18, 2008

(54) Lipoxygenase and Cyclooxygenase Inhibition

(76) Inventors: Shixin Deng, Sandy, UT (US); Brett West, Orem, UT (US); Afa Palu, American Fork, UT (US); Chen Xing Su, West Jordan, UT (US); Bing-Nan Zhou, Pleasant Grove, UT (US); Claude Jarakae Jensen, Cedar Hills, UT (US)

Correspondence Address: KIRTON AND MCCONKIE, 60 EAST SOUTH TEMPLE,, SUITE 1800, SALT LAKE CITY, UT, 84111, US

(21) Appl. No.: 11/945,727

(22) Filed: Nov. 27, 2007

Related U.S. Application Data
(60) Provisional application No. 60/867,478, filed on Nov. 28, 2006.

(51) Int. Cl. A61K 36/00; A61K 31/34; A61K 31/35; A61P 43/00

(52) U.S. Cl. 424/774; 424/725; 424/777; 514/470; 514/473; 514/457; 514/456

(57) ABSTRACT

The present invention relates to methods, compositions and their constituents for inhibiting 5-Lipoxygenase (5-LO), 15-Lipoxygenase (15-LO), cyclooxygenase-1 (COX-1) and cyclooxygenase-2 (COX-2), in living organisms. More particularly, the present invention relates to methods and compositions involving the inhibition of 5-Lipoxygenase (5-LO), 15-Lipoxygenase (15-LO), cyclooxygenase-1 (COX-1) and cyclooxygenase-2 (COX-2), using processed Morinda citrifolia L. plant products.

### RELATED APPLICATIONS

This application claims priority to provisional application Ser. No. 60/867,478 filed Nov. 28, 2006, for “Lipoxygenase and Cyclooxygenase Inhibition.”

### BACKGROUND OF THE INVENTION

1. Field of the Invention

The present invention relates to compositions comprising Morinda citrifolia and its constitutes, and methods for obtaining the same to inhibit 5-Lipoxygenase (5-LO), 15-Lipoxygenase (15-LO), cyclooxygenase-1 (COX-1) and cyclooxygenase-2 (COX-2).

2. Background Information

Lifestyle factors, such as high-fat dietary habits, appear to contribute to many diseases common in the Western world. Linoleic acid, an unsaturated omega-6 essential fatty acid found abundantly in Western high-fat diets, is the precursor of arachidonic acid. These fatty acids are the substrates for a group of bioactive lipid metabolites metabolized by various catabolic enzymes, including COX and LO. Overexpression of COX-2 and/or 5-LO and 15-LO are often observed some cancers, such as colon, stomach, breast, and lung cancers, and these enzymes appear to significantly contribute to the initiation and development of cancers. In addition, the metabolites of COX-2 and LO enzymes involve in inflammation. Therefore, 15-LO and COX-2 are regarded as significant potential molecular targets for cancer prevention, as well as anti-inflammation.

Many natural constituents from fruits and vegetables have been identified as inhibitors of COX-2 and LO enzymatic activities in vitro, exhibiting anti-inflammatory effects. For these reasons, they are often recommended for prevention of cancer and other diseases.

Morinda citrifolia L. (Rubiaceae) is a small tropical evergreen shrub or tree indigenous to the Pacific Islands, Southeast Asia, and other tropical and semi-tropical areas. In previous phytochemical studies, various compounds have been isolated and/or identified from noni fruit.

### SUMMARY OF THE INVENTION

Embodiments of the present invention relate to various methods of using specially processed components of the Indian Mulberry or Morinda citrifolia L. plant to inhibit 5-Lipoxygenase (5-LO), 15-Lipoxygenase (15-LO), cyclooxygenase-1 (COX-1) and cyclooxygenase-2 (COX-2).

Some embodiments of the invention include one or more processed Morinda citrifolia components such as: extract from the leaves of Morinda citrifolia, leaf hot water extract, processed Morinda citrifolia leaf ethanol extract, processed Morinda citrifolia leaf steam distillation extract, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice, whole Morinda citrifolia fruit in fresh, whole dried Morinda citrifolia fruit, powder or solvent extracted forms as well as enzyme treated Morinda citrifolia seeds, or any other processed Morinda citrifolia seed (i.e. roasting, blanching, microwaving, heat treatment, soaking in water or water solutions of various salts or chemical compounds), whole Morinda citrifolia fruit with blossoms or flowers attached, leaf extracts, leaf juice, and defatted and untreated seed extracts. Some of these methods include the steps of administering a Morinda citrifolia composition to a mammal to inhibit, prevent, or treat inflammatory diseases or cancer.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings in which:

FIG. 1 illustrates the new compound (+)-3,4,3′,4′-tetrahydroxy-9,7′α-epoxylignano-7α,9′-lactone (1);

FIG. 2 illustrates the new compound (+)-3,3′-bisdemethyltanegool (2);

FIG. 3 illustrates the compound pinoresinol;

FIG. 4 illustrates two flavonols quercetin (5) and kaempferol (6); and

FIG. 5 illustrates scopoletin (7) and isoscopoletin (8).

### DETAILED DESCRIPTION OF THE INVENTION

It will be readily understood that the components of the present invention, as generally described herein, could be arranged and designed in a wide variety of different configurations. Thus, the following more detailed description of embodiments of the compositions and methods of the present invention is not intended to limit the scope of the invention, as claimed, but is merely representative of the presently preferred embodiments of the invention. The scope of the invention is, therefore, indicated by the appended claims rather than by the foregoing description. All changes that come within the meaning and range of equivalency of the claims are to be embraced within their scope.

It will be appreciated by those of ordinary skill in the art that the objects of this invention can be achieved without the expense of undue experimentation using well known variants, modifications, or equivalents of the methods and techniques described herein. The skilled artisan will also appreciate that alternative means, other than those specifically described, are available in the art to achieve the functional features of the molecules described herein. It is intended that the present invention include those variants, modifications, alternatives, and equivalents which are appreciated by the skilled artisan and encompassed by the spirit and scope of the present disclosure.

Embodiments of the present invention feature methods, compositions and their components for inhibiting 5-Lipoxygenase (5-LO), 15-Lipoxygenase (15-LO), cyclooxygenase-1 (COX-1), cyclooxygenase-2 (COX-2), skin cancer, and for treating and preventing mammalian inflammatory diseases and skin cancer through the administration of a composition comprising components of the Indian Mulberry or Morinda citrifolia L. plant.

1. General Description of the Morinda citrifolia L. Plant

The Indian Mulberry or Morinda citrifolia plant, known scientifically as Morinda Citrifolia L. (“Morinda citrifolia”), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds. When fully ripe, the fruit has a pronounced odor like rancid cheese. Although the fruit has been eaten by several nationalities as food, the most common use of the Morinda citrifolia plant has traditionally been as a red and yellow dye source.

2. Processing Morinda citrifolia Leaves

The leaves of the Morinda citrifolia plant are one possible component of the Morinda citrifolia plant that may be present in some compositions of the present invention. For example, some compositions comprise leaf extract and/or leaf juice as described further herein. Some compositions comprise a leaf serum that is comprised of both leaf extract and fruit juice obtained from the Morinda citrifolia plant. Some compositions of the present invention comprise leaf serum and/or various leaf extracts as incorporated into a nutraceutical product (“nutraceutical” herein referring to any drug or product designed to improve the health of living organisms such as human beings or mammals).

In some embodiments of the present invention, the Morinda citrifolia leaf extracts are obtained using the following process. First, relatively dry leaves from the Morinda citrifolia L. plant are collected, cut into small pieces, and placed into a crushing device—preferably a hydraulic press—where the leaf pieces are crushed. In some embodiments, the crushed leaf pieces are then percolated with an alcohol such as ethanol, methanol, ethyl acetate, or other alcohol-based derivatives using methods known in the art. Next, in some embodiments, the alcohol and all alcohol-soluble ingredients are extracted from the crushed leaf pieces, leaving a leaf extract that is then reduced with heat to remove all the liquid therefrom. The resulting dry leaf extract will herein be referred to as the “primary leaf extract.”

In some embodiments of the present invention, the primary leaf extract is pasteurized to at least partially sterilize the extract and destroy objectionable organisms. The primary leaf extract is pasteurized preferably at a temperature ranging from 70 to 80 degrees Celsius and for a period of time sufficient to destroy any objectionable organisms without major chemical alteration of the extract. Pasteurization may also be accomplished according to various radiation techniques or methods.

In some embodiments of the present invention, the pasteurized primary leaf extract is placed into a centrifuge decanter where it is centrifuged to remove or separate any remaining leaf juice therein from other materials, including chlorophyll. Once the centrifuge cycle is completed, the leaf extract is in a relatively purified state. This purified leaf extract is then pasteurized again in a similar manner as discussed above to obtain a purified primary leaf extract.

Preferably, the primary leaf extract, whether pasteurized and/or purified, is further fractionated into two individual fractions: a dry hexane fraction, and an aqueous methanol fraction. This is accomplished preferably via a gas chromatograph containing silicon dioxide and CH₂Cl₂—MeOH ingredients using methods well known in the art. In some embodiments of the present invention, the methanol fraction is further fractionated to obtain secondary methanol fractions. In some embodiments, the hexane fraction is further fractionated to obtain secondary hexane fractions.

One or more of the leaf extracts, including the primary leaf extract, the hexane fraction, methanol fraction, or any of the secondary hexane or methanol fractions may be combined with the fruit juice of the fruit of the Morinda citrifolia plant to obtain a leaf serum (the process of obtaining the fruit juice to be described further herein). In some embodiments, the leaf serum is packaged and frozen ready for shipment; in others, it is further incorporated into a nutraceutical product as explained herein.

3. Processing Morinda citrifolia Fruit

Some embodiments of the present invention include a composition comprising fruit juice of the Morinda citrifolia plant. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another product, frozen or pasteurized. In some embodiments of the present invention, the juice and pulp can be pureed into a homogenous blend to be mixed with other ingredients. Other processes include freeze drying the fruit and juice. The fruit and juice can be reconstituted during production of the final juice product. Still other processes may include air drying the fruit and juices prior to being masticated.

In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, but preferably for 2 to 3 days. The fruit is ripened or aged by being placed on equipment so that the fruit does not contact the ground. The fruit is preferably covered with a cloth or netting material during aging, but the fruit can be aged without being covered. When ready for further processing the fruit is light in color, such as a light green, light yellow, white or translucent color. The fruit is inspected for spoilage or for excessive green color and firmness. Spoiled and hard green fruit is separated from the acceptable fruit.

The ripened and aged fruit is preferably placed in plastic lined containers for further processing and transport. The containers of aged fruit can be held from 0 to 30 days, but preferably the fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions. The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.). Another product manufactured is Morinda citrifolia puree and puree juice, in either concentrate or diluted form. Puree is essentially the pulp separated from the seeds and is different than the fruit juice product described herein.

The product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, a reverse osmosis filtration device, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The resulting pulp extract typically has a fiber content of 10 to 40 percent by weight. The resulting pulp extract is preferably pasteurized at a temperature of 181° F. (83° C.) minimum and then packed in drums for further processing or made into a high fiber product.

4. Processing Morinda citrifolia Seeds

Some Morinda citrifolia compositions of the present invention include seeds from the Morinda citrifolia plant. In some embodiments of the present invention, Morinda citrifolia seeds are processed by pulverizing them into a seed powder in a laboratory mill. In some embodiments, the seed powder is left untreated. In some embodiments, the seed powder is further defatted by soaking and stirring the powder in hexane—preferably for 1 hour at room temperature (Drug:Hexane—Ratio 1:10). The residue, in some embodiments, is then filtered under vacuum, defatted again (preferably for 30 minutes under the same conditions), and filtered under vacuum again. The powder may be kept overnight in a fume hood in order to remove the residual hexane.

Still further, in some embodiments of the present invention, the defatted and/or untreated powder is extracted, preferably with ethanol 50% (m/m) for 24 hours at room temperature at a drug solvent ratio of 1:2.

5. Processing Morinda citrifolia Oil

Some embodiments of the present invention may comprise oil extracted from the Morinda Citrifolia plant. The method for extracting and processing the oil is described in U.S. patent application Ser. No. 09/384,785, filed on Aug. 27, 1999 and issued as U.S. Pat. No. 6,214,351 on Apr. 10, 2001, which is incorporated by reference herein. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

### 6. Compositions and Their Use

The compositions of the present invention may be formulated into any of a variety of compositions, including orally administered compositions, intravenous solutions, and other products or compositions. As mentioned earlier herein, the compositions can include a variety of ingredients.

Compositions of the present invention may comprise any of a number of Morinda citrifolia components such as: leaf extract, leaf juice, leaf serum, fruit juice, fruit pulp, pulp extract, puree, seeds (whether defatted or untreated), and oil. Compositions of the present invention may also include various other ingredients. Examples of other ingredients include, but are not limited to: artificial flavoring, other natural juices or juice concentrates such as a natural grape juice concentrate or a natural blueberry juice concentrate; carrier ingredients; and others as will be further explained herein.

Any compositions having the leaf extract from the Morinda citrifolia leaves, may comprise one or more of the following: the primary leaf extract, the hexane fraction, methanol fraction, the secondary hexane and methanol fractions, ethanol fractions, ethyl acetate extractions, the leaf serum, or the nutraceutical leaf product.

In some embodiments of the present invention, active ingredients or compounds of Morinda citrifolia components may be extracted out using various procedures and processes commonly known in the art. For instance, the active ingredients may be isolated and extracted out using alcohol or alcohol-based solutions, such as methanol, ethanol, and ethyl acetate, and other alcohol-based derivatives using methods known in the art. These active ingredients or compounds may be isolated and further fractioned or separated from one another into their constituent parts. Preferably, the compounds are separated or fractioned to identify and isolate any active ingredients that might help to prevent disease, enhance health, or perform other similar functions. In addition, the compounds may be fractioned or separated into their constituent parts to identify and isolate any critical or dependent interactions that might provide the same health-benefiting functions just mentioned.

Any components and compositions of Morinda citrifolia may be further incorporated into a nutraceutical product (again, “nutraceutical” herein referring to any drug or product designed to improve the health of living organisms such as human beings or mammals). Examples of nutraceutical products may include, but are not limited to: intravenous products, topical dermal products, wound healing products, skin care products, hair care products, beauty and cosmetic products (e.g., makeup, lotions, etc.), bum healing and treatment products, first-aid products, antibacterial products, lip balms and ointments, bone healing and treatment products, meat tenderizing products, anti-inflammatory products, eye drops, deodorants, antifungal products, arthritis treatment products, muscle relaxers, toothpaste, and various nutraceutical and other products as may be further discussed herein.

The compositions of the present invention may be formulated into any of a variety of embodiments, including oral compositions, topical dermal solutions, intravenous solutions, and other products or compositions.

Oral compositions may take the form of, for example, tablets, lozenges, aqueous or oily suspensions, dispersible powders or granules, emulsions, syrups, or elixirs. Compositions intended for oral use may be prepared according to any method known in the art, and such compositions may contain one or more agents such as sweetening agents, flavoring agents, coloring agents, and preserving agents. They may also contain one or more additional ingredients such as vitamins and minerals, etc. Tablets may be manufactured to contain one or more Morinda citrifolia components in admixture with non-toxic, pharmaceutically acceptable excipients that are suitable for the manufacture of tablets. These excipients may be, for example, inert diluents, granulating and disintegrating agents, binding agents, and lubricating agents. The tablets may be uncoated or they may be coated by known techniques to delay disintegration and absorption in the gastrointestinal tract and thereby provide sustained action over a longer period. For example, a time delay material such as glyceryl monostearate or glyceryl distearate may be used.

Aqueous suspensions may be manufactured to contain the Morinda citrifolia components in admixture with excipients suitable for the manufacture of aqueous suspensions. Examples of such excipients include, but are not limited to: suspending agents such as sodium carboxymethyl-cellulose, methylcellulose, hydroxy-propylmethycellulose, sodium alginate, polyvinyl-pyrrolidone, gum tragacanth and gum acacia; dispersing or wetting agents such as a naturally-occurring phosphatide like lecithin, or condensation products of an alkylene oxide with fatty acids such as polyoxyethylene stearate, or condensation products of ethylene oxide with long chain aliphatic alcohols such as heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides such as polyethylene sorbitan monooleate.

Typical sweetening agents may include, but are not limited to: natural sugars derived from corn, sugar beets, sugar cane, potatoes, tapioca, or other starch-containing sources that can be chemically or enzymatically converted to crystalline chunks, powders, and/or syrups. Also, sweeteners can comprise artificial or high-intensity sweeteners, some of which may include aspartame, sucralose, stevia, saccharin, etc. The concentration of sweeteners may be between from 0 to 50 percent by weight of the Morinda citrifolia composition, and more preferably between about 1 and 5 percent by weight.

Typical flavoring agents can include, but are not limited to, artificial and/or natural flavoring ingredients that contribute to palatability. The concentration of flavors may range, for example, from 0 to 15 percent by weight of the Morinda citrifolia composition. Coloring agents may include food-grade artificial or natural coloring agents having a concentration ranging from 0 to 10 percent by weight of the Morinda citrifolia composition.

Typical nutritional ingredients may include vitamins, minerals, trace elements, herbs, botanical extracts, bioactive chemicals, and compounds at concentrations from 0 to 10 percent by weight of the Morinda citrifolia composition. Examples of vitamins include, but are not limited to, vitamins A, B1 through B12, C, D, E, Folic Acid, Pantothenic Acid, Biotin, etc. Examples of minerals and trace elements include, but are not limited to, calcium, chromium, copper, cobalt, boron, magnesium, iron, selenium, manganese, molybdenum, potassium, iodine, zinc, phosphorus, etc. Herbs and botanical extracts may include, but are not limited to, alfalfa grass, bee pollen, chlorella powder, Dong Quai powder, Ecchinacea root, Gingko Biloba extract, Horsetail herb, Indian mulberry, Shitake mushroom, spirulina seaweed, grape seed extract, etc. Typical bioactive chemicals may include, but are not limited to, caffeine, ephedrine, L-carnitine, creatine, lycopene, etc.

The ingredients to be utilized in a topical dermal product may include any that are safe for internalizing into the body of a mammal and may exist in various forms, such as gels, lotions, creams, ointments, etc., each comprising one or more carrier agents. The ingredients or carrier agents incorporated into systemically (e.g., intravenously) administered compositions may also comprise any known in the art.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

Favorably, this invention provides a method of inhibiting 5-Lipoxygenase (5-LO), 15-Lipoxygenase (15-LO), cyclooxygenase-1 (COX-1) and/or cyclooxygenase-2 (COX-2) with a Morinda citrifolia-based formulation without any significant tendency to cause undesirable side effects.

The present invention features a unique formulation and method of administering the same to inhibit 5-Lipoxygenase (5-LO), 15-Lipoxygenase (15-LO), cyclooxygenase-1 (COX-1) and/or cyclooxygenase-2 (COX-2), by providing a nutraceutical composition or treatment formulated with one or more processed Morinda citrifolia products derived from the Indian Mulberry plant. The Morinda citrifolia product is incorporated into various carriers or nutraceutical compositions suitable for in vivo treatment of a patient. For instance, the nutraceutical formulation may be ingested orally, introduced via an intravenous injection or feeding system, or otherwise internalized as is appropriate and directed.

The nutraceutical compositions of the present invention may comprise one or more of a processed Morinda citrifolia product present in an amount by weight between about 0.01 and 100 percent by weight, and preferably between 0.01 and 95 percent by weight. Several exemplary embodiments of formulations are provided below. However, these are only intended to be exemplary, as one ordinarily skilled in the art will recognize other formulations or compositions comprising the processed Morinda citrifolia product.

The processed Morinda citrifolia product may be the active ingredient or may contain one or more active ingredient, such as quercetin, rutin, scopoletin, octoanoic acid, potassium, vitamin C, terpenoids, alkaloids, anthraquinones (such as nordamnacanthal, morindone, rubiadin, alizarin), B-sitosterol, carotene, vitamin A, flavone glycosides, linoleic acid, amino acids, acubin, L-asperuloside, caproic acid, caprylic acid, ursolic acid, (tahitinin A, tahitinin B, pinoresinol, 3,3′-bisdemethylpinoresinol, quercetin, kaempferol, scopoletin, isoscopoletin, aromatic vanillin, and a putative proxeronine and others, for inhibiting 5-Lipoxygenase (5-LO), 15-Lipoxygenase (15-LO), cyclooxygenase-1 (COX-1) and cyclooxygenase-2 (COX-2). Active ingredients may be extracted utilizing aqueous or organic solvents including various alcohol or alcohol-based solutions, such as methanol, ethanol, and ethyl acetate, and other alcohol-based derivatives using any known process in the art. The active ingredients of quercetin and rutin are present in amounts by weight ranging from 0.01-10 percent of the total formulation or composition. These amounts may be concentrated as well into a more potent concentration in which they are present in amounts ranging from 10 to 100 percent.

The nutraceutical composition comprising Morinda citrifolia may be prepared using any known means in the art. In addition, since the nutraceutical composition will most likely be consumed orally, it may contain one or more agents selected from the group consisting of sweetening agents, flavoring agents, coloring agents, preserving agents, and other medicinal agents as directed.

The present invention further features a method of administering a nutraceutical composition comprising one or more processed Morinda citrifolia products to inhibit 5-Lipoxygenase (5-LO), 15-Lipoxygenase (15-LO), cyclooxygenase-1 (COX-1) and/or cyclooxygenase-2 (COX-2) by providing a nutraceutical composition or treatment. The method for administering a nutraceutical, or the method for inhibiting 5-Lipoxygenase (5-LO), 15-Lipoxygenase (15-LO), cyclooxygenase-1 (COX-1) and/or cyclooxygenase-2 (COX-2), may comprise the steps of (a) formulating a nutraceutical composition comprising in part a processed Morinda citrifolia product present in an amount between about 0.01 and 95 percent by weight, wherein the composition may also comprise a carrier, such as water or purified water, and other natural or artificial ingredients; (b) introducing the nutraceutical composition into the body, such that the processed Morinda citrifolia product is sufficiently internalized; (c) repeating the above steps as often as necessary to provide an effective amount of the processed Morinda citrifolia product to the body of the patient to inhibit 5-Lipoxygenase (5-LO), 15-Lipoxygenase (15-LO), cyclooxygenase-1 (COX-1) and/or cyclooxygenase-2 (COX-2).

The step of introducing the nutraceutical composition into the body may comprise the step of ingesting the composition orally. Ingesting the nutraceutical orally means the nutraceutical composition may be formulated as a liquid, gel, solid, or some other type that would allow the composition to be quickly digested and concentrated within the body. The step of administering the nutraceutical composition may be carried out in an effective manner so that the greatest concentration of nutraceutical composition, and particularly the processedMorinda citrifolia product, is internalized and absorbed into the patient's body. In one embodiment, the nutraceutical composition is administered by taking between 1 teaspoon and 2 oz., and preferably 2 oz., of the nutraceutical composition every two hours each day, or at least twice a day. In addition, the nutraceutical composition is to be taken on an empty stomach, meaning at a period of time at least two hours prior to consumption of any food or drink. Following this, the nutraceutical composition is sufficiently allowed to absorb into the tissues of the body. This invention contemplates that the amount of composition and frequency of use may vary from individual to individual. For example, the invention contemplates the administration of up to 10 ounces. for each administration.

In another method of the present invention, a takes at least one (1) ounce of Formulation One in the morning on an empty stomach, and at least one (1) ounce at night on an empty stomach, just prior to retiring to bed. In another method of the present invention, a person diagnosed with or experiencing depression takes at least one ounce of Formulation Two twice a day. In addition, the step of administering the nutraceutical composition may include injecting the composition into the body using an intravenous pump.

The following compositions or formulations represent some of the preferred embodiments contemplated by the present invention.

### Formulation One

| Ingredients | Percent by Weight |
|---|---|
| Morinda citrifolia fruit juice | 85-99.99% |
| Other fruit juices | 0.1-15% |

### Formulation Two

| Ingredients | Percent by Weight |
|---|---|
| Morinda citrifolia fruit juice | 50-90% |
| Water | 0.1-50% |
| Other fruit juices | 0.1-30% |

### Formulation Three

| Ingredients | Percent by Weight |
|---|---|
| Morinda citrifolia extract | 100% |

### Formulation Four

| Ingredients | Percent by Weight |
|---|---|
| Morinda citrifolia juice | 50-90% |
| Morinda citrifolia extract | 0.1-50% |

### Formulation Five

| Ingredients | Percent by Weight |
|---|---|
| Morinda citrifolia extract | 50-90% |
| Other fruit juices | 0.1-30% |

### Formulation Six

| Ingredients | Percent by Weight |
|---|---|
| Morinda citrifolia juice | 50-90% |
| Morinda citrifolia extract | 0.1-50% |
| Other fruit juices | 0.1-30% |

### Formulation Seven

| Ingredients | Percent by Weight |
|---|---|
| Morinda citrifolia extract | 0.1-50% |
| Water | 50-99.9% |

### 7. EXAMPLES

The following examples illustrate some of the preventative and treatment effects of some Morinda citrifolia compositions of the present invention on 5-Lipoxygenase (5-LO), 15-Lipoxygenase (15-LO), cyclooxygenase-1 (COX-1), cyclooxygenase-2 (COX-2), inflammatory diseases, and/or cancer. These examples are not intended to be limiting in any way, but are merely illustrative of benefits, advantages, and remedial effects of some embodiments of the Morinda citrifolia compositions of the present invention.

### Example One

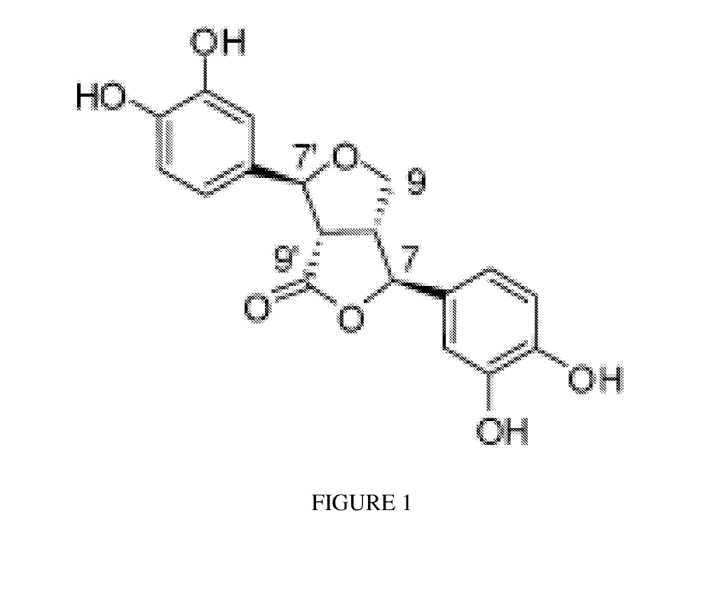

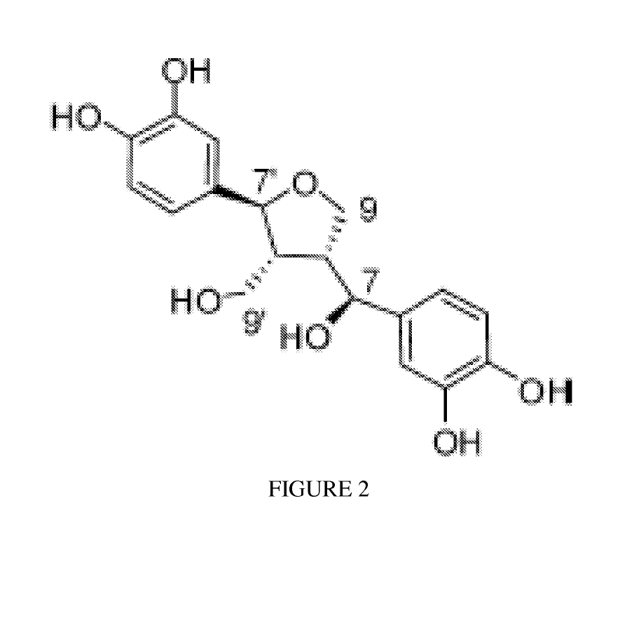

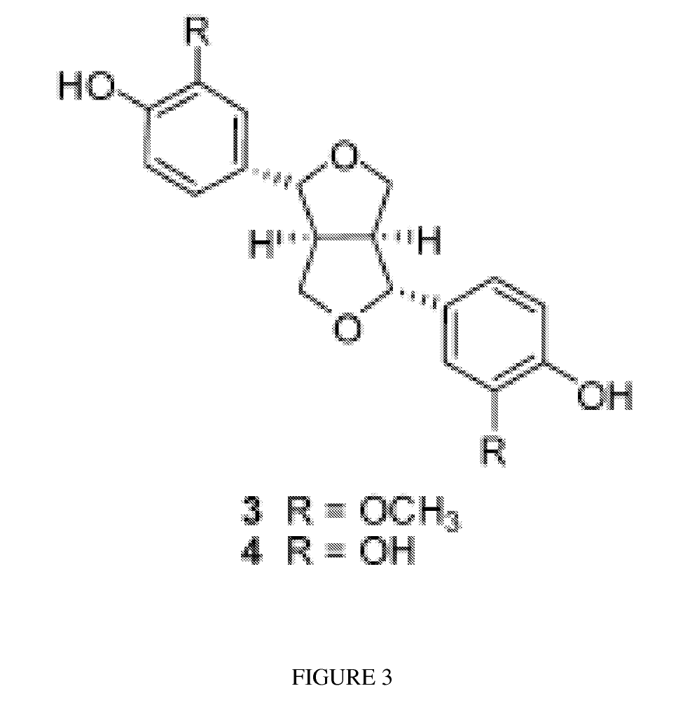

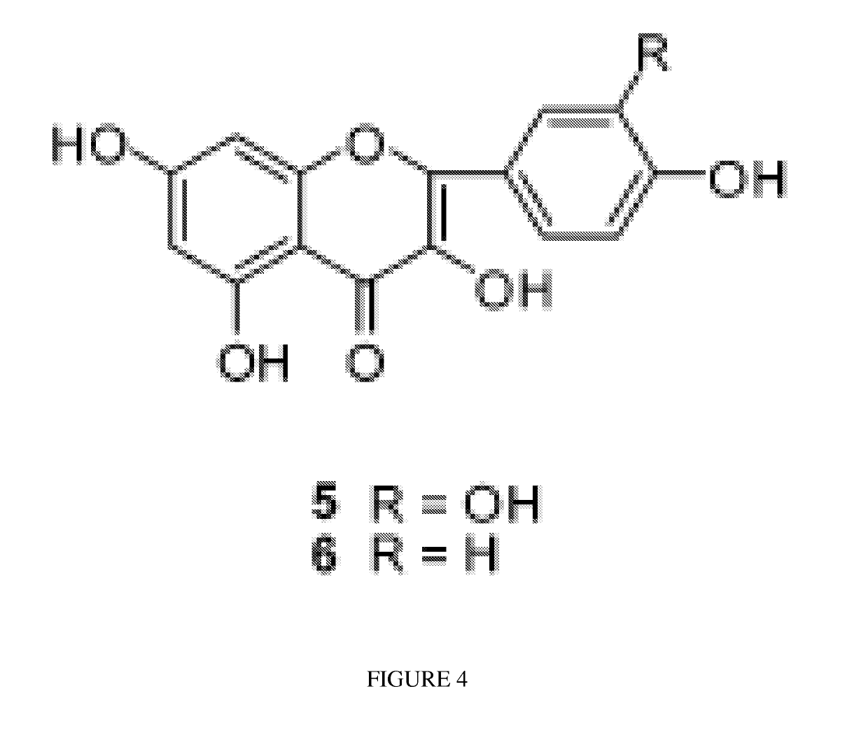

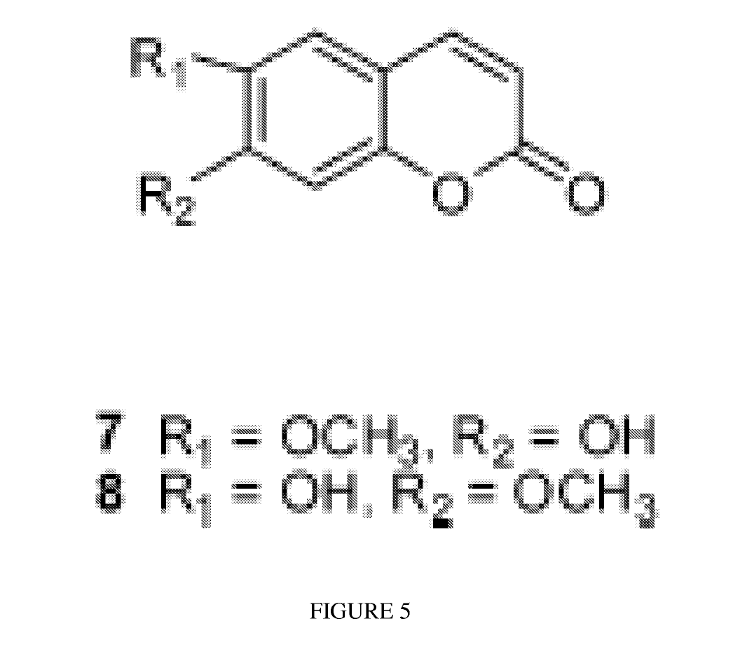

The cycloogygenase (COX-2) assay-guided phytochemical study of a methanol extract of processed fruit of Morinda citrifolia led to the production and identification of 9 compounds including two new lignans compound (1) and compound (2))(FIGS. 1 and 2), as well as seven known compounds: two lignans (pinoresinol (3)(FIG. 3) and 3,3′-bisdemethylpinoresinol (4)), two flavonols (quercetin (5) and kaempferol (6)) (FIG. 4), two coumarins (scopoletin (7) and isoscopoletin (8)) (FIG. 5), and an aromatic vanillin (9). The structures of the new compounds were determined by spectroscopic techniques including 1D and 2D NMR, and HRESIMS.

The screening of LO/COX-2 inhibitors from the active EtOAc partition of the processed M. citrifolia fruits was carried out by a series of chromatographic techniques and led to the production of two new lignans, (+)-3,4,3′,4′-tetrahydroxy-9,7′α-epoxylignano-7α,9′-lactone (1) and (+)-3,3′-bisdemethyltanegool (2), as well as seven known compounds (FIGS. 3-5). The structures of compounds 1 and 2 (FIGS. 1 and 2) were elucidated by 1D and 2D NMR spectroscopy and HRESIMS. The inhibitory effects of all nine isolates on COX-2 and COX-1, as well as 5-LO and 15-LO enzymes were evaluated in in vitro assays.

Compound 1 (FIG. 1) was produced as a light brown resinous semi-solid. Its UV spectrum revealed the presence of an aromatic ring (230 and 282 nm). The IR spectrum exhibited characteristic absorption bands at 3400, 1750, 1675 and 1520 cm⁻¹, suggesting the presence of hydroxyl, an α,β-unsaturated ester carbonyl, and aromatic moieties, respectively. The ¹H and ¹³C NMR spectral data of compound 1 (Table 1), exhibited an oxygenated methylene at δ 4.26 (1H, dd, J=9.4, 7.0 Hz) and 3.98 (1H, dd, J=9.4, 4.2 Hz), two oxygenated methine at δ 5.31 (1H, d, J=3.6 Hz) and 5.16 (1H, d, J=3.4 Hz), together with two methane at δ 3.26 (1H, m) and 3.58 (1H, dd, J=9.0, 3.4 Hz).

The HRESIMS spectrum displayed a protonated molecule of m/z 345.0954 (calcd for m/z 345.0968), corresponding to a molecular formula of C₁₈H₁₆O₇, containing eleven degrees of unsaturation. In addition, the presence of two sets of 1,3,4-trisubstituted phenyl units (ABX system) was indicated by signals at δ 6.77 (1H, d, J=7.9 Hz), δ 6.69 (1H, dd, J=7.9, 2.0 Hz) and δ 6.76 (1H, d, J=2.0 Hz), as well as those at δ 6.82 (1H, d, J=1.8 Hz), δ 6.74 (1H, d, J=7.8 Hz) and δ 6.72 (1H, dd, J=7.8, 1.8 Hz), and confirmed by the COSY spectrum. The COSY spectrum of compound 1 revealed the presence of a moiety of —CH₂—CH(CH)—CH—CH—, which corresponds to positions C-7, 8, 9, 7′ and 8′ in a dioxabicyclo[3.3.0] octane skeleton. Its ¹³C and DEPT NMR spectra indicated the presence of eighteen carbons in the molecule, including an ester carbonyl group (δ 179.9), two oxygenated methine groups (δ 87.2 and δ 85.2), another two non-oxygenated methine (δ 52.0 and δ 54.5), as well as twelve aromatic carbons at the range of δ 114.0 and δ 147.0 in the downfield region. The 1 H and 13 C NMR spectroscopic data of compound 1 (Table 1) resembled those of graminone A. Further analysis of the observed 2D NMR spectra (¹H-¹H COSY, HSQC and HMBC) fully established the connectivity of the molecule.

In the HMBC spectra, the following key correlations were observed: H-7→C-2, C-6, C-9, C-8′ and C-9′; H-8→C-1, C-7′ and C-9′; H-9→C-7, C-8′; H-7′→C-8, C-9, C-2′, C-6′ and C-9′; H-8′→C-7, C-9 and C-1′) (Table 1).

TABLE 1
¹H and ¹³C NMR Spectroscopic Data (500 Mz, CD₃OD) for Isolates 1 and 2^{a}

| | 1 | | | 2 | | |
|---|---|---|---|---|---|---|
| position | δ_{C} | δ_{H} (J in Hz) | HMBC^{b} | δ_{C} | δ_{H} (J in Hz) | HMBC^{b} |
| 1 | 132.7 | | 5, 7, 8 | 130.2 | | 5, 7, 8 |
| 2 | 114.0 | 1H, 6.76, d (2.0) | 6, 7 | 112.9 | 1H, 6.82, d (1.8) | 6, 7 |
| 3 | 147.1 | | 5 | 145.0 | | 5 |
| 4 | 146.7 | | 2, 6 | 144.1 | | 2, 6 |
| 5 | 116.6 | 1H, 6.77, d (7.9) | | 115.0 | 1H, 6.74, d (8.1) | |
| 6 | 118.7 | 1H, 6.69, dd (7.9, 2.0) | 2, 7 | 116.9 | 1H, 6.68, dd (8.1, 1.8) | 2 |
| 7 | 87.2 | 1H, 5.31, d (3.6) | 2, 6, 9, 8′ | 82.3 | 1H, 4.80, d (6.0) | 2, 9, 9′ |
| 8 | 52.0 | 1H, 3.26, m | 7, 9, 7′, 8′ | 50.1 | 1H, 3.34, m | 9′ |
| 9 | 73.9 | Ha: 4.26, dd (9.4, 7.0); | 7, 7′, 8′ | 69.4 | Ha: 3.77, dd (8.3, 8.3); | 7 |
| | | Hb: 3.98, dd (9.4, 4.2) | | | Hb: 3.29, dd (8.3, 8.3) | |
| 1′ | 133.5 | | 5′, 7′, 8′ | 132.6 | | 5′, 7′, 8′ |
| 2′ | 114.2 | 1H, 6.82, d (1.8) | 7′ | 113.3 | 1H, 6.89, d (1.8) | 7′ |
| 3′ | 147.0 | | 5′ | 145.0 | | 5′ |
| 4′ | 146.4 | | 2′, 6′ | 144.6 | | 2′, 6′ |
| 5′ | 116.4 | 1H, 6.74, d (7.8) | | 114.9 | 1H, 6.73, d (7.8) | |
| 6′ | 118.4 | 1H, 6.72, dd (7.8, 1.8) | 2′, 7′ | 117.8 | 1H, 6.69, dd (7.8, 1.8) | 2′, 7′ |
| 7′ | 85.2 | 1H, 5.16, d (3.4) | 8, 9, 2′, 6′ | 88.2 | 1H, 4.33, d (6.7) | 2′, 9′ |
| 8′ | 54.5 | 1H, 3.58, dd (9.9, 3.4) | 7, 8, 9, 7′ | 54.9 | 1H, 2.89, ddd (8.2, 6.7, 6.7) | 9 |
| 9′ | 179.9 | | 7, 8, 7′, 8′ | 70.7 | Ha: 4.05, d (9.4); | 7, 7′ |
| | | | | | Hb: 3.82, dd (9.4, 6.7) | |

^{a}Chemical shifts are shown in δ values (ppm) relative to TMS. Assignments and determination of multiplicities were aided by 2D NMR (COSY, HMQC and HMBC).
^{b}HMBC correlations are from proton(s) to the indicated carbons.

The relative stereochemistry of compound 1 was established by the analysis of coupling constants in the ¹H and NOESY spectral data. The coupling constants of H-7 (J=3.6 Hz) and H-7′ (J=3.4 Hz) suggested that axial protons exist at these positions. The NOESY spectrum of compound 1 exhibited the correlations between H-8 and H-8′, H-7 and H-9b, as well as H-8 and H-9a, which indicates that these protons are on the same face of the molecule, and confirm the relative configuration proposed (FIG. 1). On the basis of above-mentioned data, it was identified as a previously unreported lignan. According to the nomenclature of lignans and neolignans recommended by International Union of Pure and Applied Chemistry (IUPAC) in 2000, compound 1 was elucidated as 3,3′,4,4′-tetrahydroxy-7′α,9-epoxylignano-7α,9′-lactone. It was given the trivial name of tahitinin A, as it was isolated for the first time from noni fruit collected from the Tahitian islands.

Compound 2 was isolated as a greenish brown resinous semi-solid. Its spectroscopic data (UV, IR, ¹H and ¹³C NMR) were very similar to those of compound 1, suggesting it could be another lignan. Its molecular formula of C₁₈H₂₀O₇ was established on the basis of a protonated molecular ion peak at m/z 349.1265 in the HRESIMS spectrum (calcd for C₁₈H₂₁O₇, 349.1281).

The ¹H NMR spectrum of compound 2 displayed two oxygenated methylene at H-9 [δ 3.77 (1H, dd, J=8.3, 8.3 Hz) and 3.29 (1H, dd, J=8.3, 8.3 Hz)] and H-9′ [δ 4.05 (1H, d, J=9.4 Hz) and 3.82 (1H, dd, J=9.4, 6.7 Hz)], two oxygenated methine at H-7 [δ 4.80 (1H, d, J=6.0 Hz)] and H-7′ [δ 4.33 (1H, d, J=6.7 Hz)], the other two methine at H-8 [δ 3.34 (1H, m)] and H-8′ [δ 2.89 (1H, ddd, J=8.2, 6.7, 6.7 Hz)], as well as two 1,3,4-trisubstituted benzenic rings (ABX system) [δ 6.82 (1H, d, J=1.8 Hz), δ 6.74(1H, d, J=8.1 Hz) and 6.68 (1H, dd, J=8.1, 1.8 Hz); δ 6.80 (1H, d, J=1.8 Hz), 6.73 (1H, d, J=7.8 Hz) and 6.69 (1H, dd, J=7.8, 1.8 Hz)].

By comparing the ¹H spectra of compounds 1 and 2, we found the addition of two more oxygenated methylene proton signals at H-9′ [δ 4.05 (1H, d, J) 9.4 Hz) and 3.82 (1H, dd, J) 9.4, 6.7 Hz)] in 2. Further comparison of the ¹³C spectra of 1 and 2 indicated no carbonyl signal in 2. The COSY spectrum suggested the presence of —CH₂—CH(CH)—CH(CH)—CH₂— as partial structure in the molecule of compound 2. The gross structure of compound 2 was fully elucidated by the HMBC spectrum with the key correlations between H-7 and C-2, C-5, C-9, C-8′ and C-9′, H-7′ and C-2′, C-6′ and C-9′, as well as H-9′ and C-8 (Table 1). The relative configuration of 2 was revealed by the correlations between H-8 and H-8′, H-8 and H-9a, and H-7′ and H-9b, observed in the NOESY spectrum (FIG. 5). Its structure was further confirmed by comparison with tanegool, a 3,3′-dimethoxylated derivative of 2. Accordingly, compound 2 was recognized as a new compound, 7,7′-epoxylignan-3,3′,4,4,9,7′-hexaol, and trivially named tahitinin B.

In addition to these new compounds 1 and 2, seven previously known compounds (3-9), were identified by comparison of their spectral data with published literature, and/or with those of authentic samples as scopoletin, kaempferol, isoscopoletin, quercetin, pinoresinol, 3,3′-bisdemethylpinoresinol, and vanillin; of which, isoscopoletin, quercetin, kaempferol and pinoresinol were isolated for the first time from noni fruit.

All of the compounds were evaluated for their anti-inflammatory effects in terms of their ability to inhibit activities of COX/LO enzymes in the in vitro assays, as summarized in Table 2.

TABLE 2
Inhibitory Activities against the 5-LO, 15-LO, and COX-2
Enzymes of Isolates 1-8 from the Fruits of M. citrifolia

| | IC₅₀^{a}(μM) | | |
|---|---|---|---|
| compound | 5-LO | 15-LO | COX-2 |
| 1 | 5.6 | 0.52 | 90.2 |
| 2 | 9.2 | 0.76 | 73.9 |
| 3 | 13.8 | 3.5 | >100 |
| 4 | 5.9 | 0.52 | >100 |
| 5 | 0.79 | 0.43 | 28.6 |
| 6 | 2.7 | 2.2 | >100 |
| 7 | >100 | 16.5 | >100 |
| 8 | >100 | 15.1 | >100 |
| nordihydroguaiaretic acid^{b} | 0.13 | | |
| PD-146176^{b} | | 1.12 | |
| rofecoxib^{b} | | | 0.12 |

^{a}IC₅₀ values represent concentration (μM) of samples to inhibit enzyme activities by 50%. Data represent average ± SD of triplicate determinations;
^{b}Positive controls.

Compounds 1, 2 and 5 showed weak inhibitory effects on the COX-2 enzyme activity, with an IC₅₀ value of 90.2, 73.9, and 28.6 microM, respectively. In the 5-LO assay, compounds 1-4, and 6, were found to inhibit 5-LO enzyme with IC₅₀ values ranging from 2.65 to 13.8 μM. Meanwhile, compound 5 displayed a potent inhibitory activity toward 5-LO enzyme, displaying an IC₅₀ of 0.79 μM. In the 15-LO assay, a moderate inhibitory effect to 15-LO enzyme was observed for compounds 3, 6 and 7 with IC₅₀ values from 2.15 to 16.5 μM. Very interestingly, compounds 1, 2, 4 and 5 were shown to be potent 15-LO inhibitors with their IC₅₀ values of 0.52, 0.76, 0.52, and 0.43 μM, respectively. In addition, no significant inhibition on COX-1 and nitric oxide synthase (iNOS) enzymes was observed in any of the isolates. These findings indicate that compounds produced from processed noni fruit contains dual inhibitors of COX-2 and 5-LO and 15-LO enzymes, which may at least partially contribute to the anti-inflammatory effects of noni fruit. It may also provide a rationale for its use in the cancer prevention and other inflammatory conditions.

Our preliminary experiments indicated that the MeOH extract of the noni fruit exhibited selective inhibitory activity against COX-2 enzyme (43% vs 15% inhibition of COX-2 and COX-1 at 100 μg/mL, respectively) (Table 1). A further phytochemical study revealed that the activity localizes at the ethyl acetate (EtOAc) partition (85% vs 0% inhibition of COX-2 and COX-1 at 100 μg/mL, respectively), among different partitions of the MeOH extract (Table 1).

General Experimental Procedures. The [α]_{D} values were measured on a Perkin Elmer 241 polarimeter at 20° C. The UV λₘₐₓ values were measured from HPLC-PDA chromatograms, and IR spectra were taken on a Thermo Nicolet Avatar 360 FT-IR Spectrometer. All ¹H NMR and ¹³C NMR data were recorded on Varian Inova-500 spectrometers using CDCl₃ or CD₃OD as solvents, and tetramethylsilane (TMS) as an internal standard. Chemical shifts (6) were expressed in ppm with reference to TMS signals. The ¹H-¹H COSY, HSQC, HMQC, NOESY and HMBC experiments were performed using standard pulse sequences provided by vendors. High-resolution mass spectra (HRMS) were obtained on an Agilent 1100 Series Liquid Chromatograph/Mass Selective Detector (LC/MSD) Time of Flight (TOF) mass spectrometer (Agilent Technologies, Inc., USA), equipped with an electrospray ion source (ESI). The spectrometer was operated in a positive ion mode. Vacuum liquid chromatography (VLC) was carried out on Merck silica gel 60 (200-400 mesh). Preparative HPLC was performed with a Waters Alliance™ 2690 separations module coupled to a Waters 2996 photodiode array (PDA) detector, utilizing a Waters XTerra® preparative MS C18 OBD column (10 μm, 19×300 mm, made in Ireland) at a flow rate of 5 mL/min or 6 mL/min. The Analytical thin-layer chromatography (TLC) was carried out on Merck TLC plates (250 μm thickness, KGF Si gel 60 and KGF RP-18 Si gel 60), and compounds were visualized by spraying the dried plates with p-anisaldehyde-H₂SO₄—EtOH (1:1:48), followed by heating at 110° C.

Plant materials. M. citrifolia fruit was collected from the Tahitian islands and identified by the Quality Control Department of Tahitian Noni International Inc. (TNI). A reference sample was deposited in TNI R&D lab (lot # 52410). The fresh juice of M. citrifolia was dried by lyophilizer.

Extraction and Isolation. The freeze-dried powder of M. citrifolia fruit (2 kg, lot number 52410) was percolated with 20L of methanol. After evaporation of the solvent, methanolic extract was added with 3 L of H₂O, then partitioned sequentially against petroleum ether (PE, 3000 mL×4), EtOAc (3000 mL×3), and butanol (BuOH, 2000 mL×3). The resulting PE, EtOAc, and BuOH partitions were dried in vacuo by rotary evaporation. The aqueous mother liquid was lyophilized to afford a dried aqueous partition. The MeOH extract and its partitions were evaluated for their COX-2 and COX-1 inhibitory activities in vitro.

The COX-2 active EtOAc partition (30 g) was subjected to flash column chromatography (1 kg of silica gel, 200-400 mesh), eluting with a stepwise gradient solvent system of dichloromethane-methanol (CH₂Cl₂—MeOH) (98:2→0:100), to afford thirteen primary pooled fractions (F1-F13). The primary fraction F9 (920 mg, COX-2 inhibition: 96% at 100 μg/mL) was chromatographed over a lipophilic Sephadex LH-20 (180 g), eluting with isocratic MeOH to give six secondary fractions (F9-1 to F9-6). The secondary fraction F9-5 (41 mg, COX-2 activity: 94% at 100 μg/mL), was further separated by reverse-phase preparative HPLC eluting with an isocratic solvent system of MeCN—MeOH—H₂O (15:15:70) at a flow rate of 6 mL/min, yielding compounds 1 (5.5 mg, t_{R}=26.5 min), 2 (4.0 mg, t_{R}=31.0 min), and 3 (6.4 mg, t_{R}=36.0 min). The primary fraction F2 (500 mg, COX-2 inhibition: 97% at 100 μg/mL), was fractionated over a lipophilic Sephadex LH-20 (180 g) to give four secondary fractions F2-1 to F2-4. Further purification of the F2-3 (180 mg, COX-2 activity: 89% at 100 μg/mL) was accomplished by reverse-phase preparative HPLC (isocratic 55% MeOH in H₂O, 5 mL/min), resulting in the isolation of compounds 4 (3.5 mg, t_{R}=18.2 min), 7 (5.6 mg, t_{R}=10 min), 8 (26.6 mg, t_{R}=14.8 min), and 9 (4.0 mg, t_{R}=20.1 min). Compound 5 (77 mg) was precipitated from the primary fraction F8 at room temperature. Compound 6 (9.4 mg) was obtained from the primary fraction F4 after further purification over a lipophilic Sephadex LH-20 (180 g), then eluted with pure MeOH.

In vitro Bioassays

COX-1 and COX-2 assays: COX-1 and -2 Inhibition assays were performed by the methods described previously with minor modifications. COX-2 enzyme was purchased from Sigma (Catalog # C-0858), and the PGE2 EIA kit from Amersham (Catalog # RPN 220). Human recombinant COX-2, expressed in insect Sf21 cells, and human platelets, respectively, were used in the COX-2 and COX-1 assays. The extract and partitions were dissolved in DMSO (Sigma, USA), and tested at 100 μg/mL (final concentration). The pure compounds were initially tested at 30 μM. The IC₅₀ values were determined if their activities were over 50% in the preliminary experiments. Test samples were preincubated with 0.12 μg/ml enzyme in modified Tris-HCl buffer pH 7.7 (COX-2), and with cells (5×10⁷/ml), in modified HEPES buffer pH 7.4 (COX-1) for 15 minutes at 37° C. The reactions were initiated by addition of 0.3 μM arachidonic acid (COX-2), and 100 μM arachidonic acid (COX-1), respectively, for another 5-minute incubation period, then terminated by further addition of 1 N HCl. An aliquot was then combined with the EIA kit for spectrophotometric determination of the quantity of PGE2 formed. Rofecoxib was used as a positive compound.

5-LO and 15-LO assays: Human peripheral blood mononuclear cells (PBMC) cells and rabbits reticulocytes were used in the 5-LO and 15-LO bioassays, and the assays were conducted according to the protocols reported previously, with either linoleic acid or arachidonic acid as substrates. LO activity was measured by quantifying immunodetectable Leukotriene B4 (LTB4) specifically with enzyme immunoassay (EIA) in the 5-LO assay, or monitoring 13-hydroperoxy-9,11-octadecadienoic acid (13-HPODE) spectrophotometrically in the 15-LO assay. Nordihydroguaiaretic acid, and PD-146176, were used as positive controls in the 5-LO and 15-Lo assays, respectively.

The results were expressed as either percentage inhibition, or IC₅₀, for test samples and data represent the average ±SD of at least triplicate determinations.

Tahitinin A/Compound (1): light brown resinous semisolid; [α]_{D}²⁰ 0° (c 0.1, MeOH); UV (LC-PDA) λₘₐₓ 230, 282 nm; IR νₘₐₓ (CH₂Cl₂) 3400 (broad) (OH), 2920, 1750 (CO₂R), 1675, 1520 (Ph), 1420, 1162, 1030, 830 cm⁻¹; ¹H NMR (CD₃OD, 500 MHz) and ¹³C NMR (CD₃OD, 125 MHz), (Table 2); HRESIMS [M+H]⁺ 345.0968 m/z calcd for C₁₈H₁₇O₇ (error: −1.4795 mDa).

Tahitinin B/Compound (2): greenish brown resinous semisolid; [α]_{D}²⁰ 0° (c 0.1, MeOH); UV (LC-PDA) λₘₐₓ 227, 279 nm; IR νₘₐₓ (CH₂Cl₂) 3404, 2922, 1675, 1580, 1435, 1270, 1171 cm⁻¹; ¹H NMR (CD₃OD, 500 MHz) and ¹³C NMR (CD₃OD, 125 MHz), (Table 2); HRESIMS [M+H]⁺ 349.1265 m/z calcd for C₁₈H₂₁O₇ (error −1.6797 mDa).

### Example Two

In the present example, a patient experiencing or diagnosed with and suffering from an inflammatory disease to treat the condition with a nonprescription, over-the-counter preparation. To treat the disease, the individual consumes an identified prescribed amount of a composition containing processed Morinda citrifolia product or extract. The person intermittently consumes the product or extract until 5-Lipoxygenase (5-LO), 15-Lipoxygenase (15-LO), cyclooxygenase-1 (COX-1) and/or cyclooxygenase-2 (COX-2) is inhibited, wherein the inflammatory disease is reduced or eliminated.

### Example Three

In the present example, a patient experiencing or diagnosed with and suffering from cancer desires to treat the condition with a nonprescription, over-the-counter preparation. To treat the disease, the individual consumes an identified prescribed amount of a composition containing processed Morinda citrifolia product or extract. The person intermittently consumes the food product containing the processed Morinda citrifolia product or extract until 5-Lipoxygenase (5-LO), 15-Lipoxygenase (15-LO), cyclooxygenase-1 (COX-1) and/or cyclooxygenase-2 (COX-2) is sufficiently inhibited, wherein the cancer is reduced or eliminated.

What is claimed is:

1. A formulation adapted to inhibit 5-lipoxygenase (5-LO), 15-Lipoxygenase (15-LO), cyclooxygenase-1 (COX-1) and cyclooxygenase-2 (COX-2) comprising: at least one processed Morinda citrifolia product present in an amount by weight between about 0.1 and 99 percent and at least one compound selected from a group consisting of (+)-3,4,3′,4′-tetrahydroxy-9,7′α-epoxylignano-7α,9′-lactone and (+)-3,3′-bisdemethyltanegool.

2. The formulation of claim 1, wherein said Morinda citrifolia product is used with a carrier medium.

3. The formulation of claim 1, wherein said processed Morinda citrifolia product comprises a processed Morinda citrifolia product selected from a group comprising: extract from the leaves of Morinda citrifolia, leaf hot water extract present in an amount by weight between about 0.1 and 50 percent, methanol extract present in an amount between 0.1 and 50 percent, a ethyl acetate extract present in an amount between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

4. The formulation of claim 1, further comprising an active ingredient selected from a group comprising quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, and putative proxeronines.

5. The formulation of claim 1, wherein said formulation is administered to a patient by a method selected from a list comprising orally, intravenously, and systemically.

6. The formulation of claim 1, further comprising an ingredient selected from the group comprising processed Morinda citrifolia products, food supplements, dietary supplements, other fruit juices, other natural ingredients, natural flavorings, artificial flavorings, natural sweeteners, artificial sweeteners, natural coloring, and artificial coloring.

7. A formulation adapted to inhibit 5-Lipoxygenase (5-LO), 15-Lipoxygenase (15-LO), cyclooxygenase-1 (COX-1) and cyclooxygenase-2 (COX-2) comprising a compound with the structural formula selected from a group consisting of:

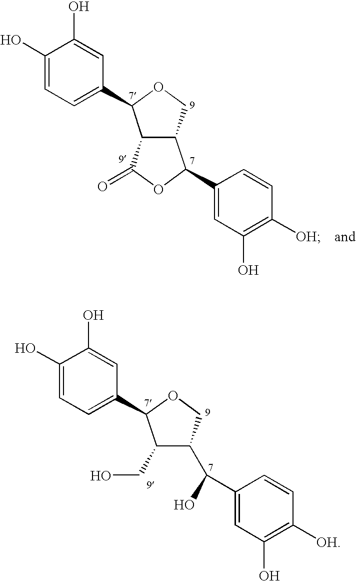

8. The formulation of claim 7, further comprising a compound with the structural formula selected from a group consisting of:

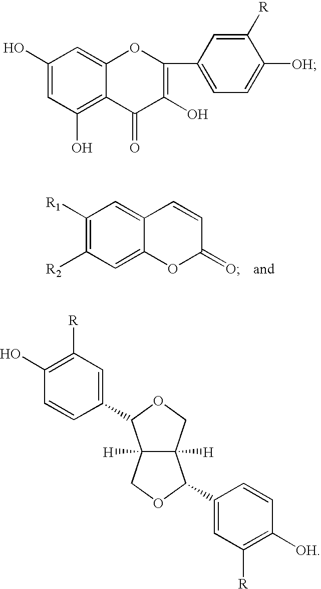

9. A method to inhibit 5-Lipoxygenase (5-LO), 15-Lipoxygenase (15-LO), cyclooxygenase-1 (COX-1) and cyclooxygenase-2 (COX-2) in mammals comprising the step of:
administering a formulation containing at least one processed Morinda citrifolia product present in an amount by weight between about 0.1 and 99 percent, wherein said processed Morinda citrifolia product comprises at least one compound selected from a group consisting of (+)-3,4,3′,4′-tetrahydroxy-9,7′α-epoxylignano-7α,9′-lactone and (+)-3,3′-bisdemethyltanegool.

10. The method of claim 9, wherein said processed Morinda citrifolia product comprises a processed Morinda citrifolia selected from a group consisting of: extract from the leaves of Morinda citrifolia, leaf hot water extract present in an amount by weight between about 0.1 and 50 percent, methanol extract present in an amount between about 0.1 and 50 percent, an ethyl acetate extract present in an amount between about 0.1 and 50 percent, processed Morinda citrifolia leaf ethanol extract present in an amount by weight between about 0.1 and 50 percent, processed Morinda citrifolia leaf steam distillation extract present in an amount by weight between about 0.1 and 50 percent, Morinda citrifolia fruit juice, Morinda citrifolia extract, Morinda citrifolia dietary fiber, Morinda citrifolia puree juice, Morinda citrifolia puree, Morinda citrifolia fruit juice concentrate, Morinda citrifolia puree juice concentrate, freeze concentrated Morinda citrifolia fruit juice, and evaporated concentration of Morinda citrifolia fruit juice.

11. The method of claim 9, wherein the formulation comprises at least one active ingredient selected from a group consisting of quercetin, rutin, scopoletin, octoanoic acid, potassium, vitamin C, terpenoids, alkaloids, anthraquinones, nordamnacanthal, morindone, rubiandin, B-sitosterol, carotene, vitamin A, flavone glycosides, linoleic acid, Alizarin, amino acids, acubin, L-asperuloside, caproic acid, caprylic acid, ursolic acid, tahitinin A, tahitinin B, pinoresinol, 3,3′-bisdemethylpinoresinol, quercetin, kaempferol, scopoletin, isoscopoletin, aromatic vanillin, and putative proxeronines.

12. The method of claim 9, wherein the formulation further comprising at least one other ingredient selected from the group consisting of processed Morinda citrifolia products, food supplements, dietary supplements, other fruit juices, other natural ingredients, natural flavorings, artificial flavorings, natural sweeteners, artificial sweeteners, natural coloring, and artificial coloring.

13. The method of claim 9, further comprising the step of concurrently administering said formulation with another medication designed to improve lipoxeganse and cyclooxygenase inhibition and associated conditions, wherein said formulation increases the efficacy of said medication.
